(19) United States
(12) Patent Application Publication
(10) Pub. No.: US 2022/0176091 A1
(43) Pub. Date: Jun. 9, 2022

(54) Ketone Oral Transfer Device

(71) Applicant: Michael S. Evans, San Antonio, TX (US)

(72) Inventor: Michael S. Evans, San Antonio, TX (US)

(21) Appl. No.: 17/248,012

(22) Filed: Jan. 5, 2021

Related U.S. Application Data
(60) Provisional application No. 63/199,055, filed on Dec. 4, 2020.

(51) Int. Cl. A61M 31/00

(52) U.S. Cl. CPC A61M 31/00

(57) ABSTRACT

The Ketone Oral Transfer Device is a ketone oral transfer device for making ketones available for ingestion or absorption of ketones, and/or provide energy form of ketones. The preferred embodiment of the device of the present invention is a liquid permeable ketone oral transfer device containing liquid soluble ketones that can be placed between the cheek and gums for the purpose of enabling ingestion or absorption of said ketones for dietary consumption, or to enable an increase in energy. An alternative embodiment of the device of the present invention is a liquid soluble membrane in the form of a flat solid strip containing liquid soluble ketones that can be placed between the cheek and gums, or on the tongue for the purpose of enabling ingestion or absorption of said ketones for dietary consumption, or to enable an increase in energy.

### TECHNICAL FIELD OF THE INVENTION

This invention relates generally to a Ketone Oral Transfer Device in the form of an orally retainable carrier comprising an edible ketone. The device helps a person who requires gradual ingestion or absorption of ketones. The person may benefit from energy occurring with metabolization of ketones.

This invention relates specifically to a liquid permeable ketone oral transfer device containing ketones that can be placed between the cheek and gums for the purpose of ingesting or absorbing the ketones to enable an increase in energy. Further, the present invention relates specifically to a liquid soluble membrane in the form of a flat strip containing ketones that can be placed between the cheek and gums, or on the tongue for the purpose of ingesting or absorbing the ketones to enable an increase in energy.

This invention relates specifically to a re-sealable and re-fillable liquid permeable ketone oral transfer device containing ketones that can be placed between the cheek and gums for the purpose of ingesting or absorbing the ketones to enable an increase in energy.

### BACKGROUND

Ketones are metabolized naturally from body fat. They provide fuel for muscles. Exogenous and oral ketone supplements are sometimes taken to enhance a person's diet. Typically, edible ketone supplements are mixed with drink or food. However, some people find the taste and/or texture of the food is less appetizing when mixed with the supplement. Also, the solubility of the edible ketone supplements in aqueous liquids is limited. Further, some people do not like to take supplement pills.

In light of the foregoing prior art, there is a need for a convenient, easy to use, store, and transport ketone oral transfer device for the purpose of enabling ingestion of ketones, enabling making ketones available for absorption, and/or to provide a source of energy in the form of ketones.

### BRIEF SUMMARY OF THE INVENTION

According to a first aspect of the invention there is a Ketone Oral Transfer Device comprising an orally retainable carrier comprising an edible ketone arranged to seep out of the carrier in oral saliva. The device makes edible ketones available for ingestion or absorption, and gradually provides energy from ketones to a user.

Preferably the carrier is permeable to oral saliva. The carrier may comprise a pouch which contains the edible ketone particles stored inside. The carrier may be retainable in a person's mouth. In this way, the ketone oral transfer device gradually delivers an edible ketone dissolved in saliva through the carrier. A person may hold the carrier on their tongue or put it between the cheek and gum. In this way, the person ingests or absorbs the ketones dissolved in saliva. The person may benefit from an increase in energy via a dose of ketones since ketones are fuel for muscles and other tissue.

A preferred form of the device is a solid strip liquid permeable ketone oral transfer device containing liquid-saliva-soluble ketones that can be placed on the tongue or between the cheek and gums for the purpose of ingesting or absorbing the ketones. As the ketones gradually seep through, a person gets a sustained supply of edible ketones to increase energy gradually over a sustained period.

An alternative embodiment of the device is the saliva permeable carrier which comprises a liquid soluble and liquid permeable membrane in the form of a solid strip or lozenge containing liquid soluble ketones. The solid strip or lozenge can be placed between the cheek and gums, or on the tongue, for the purpose of ingesting or absorbing the ketones to enable an increase in energy. The solid strip or lozenge itself preferably comprises the edible ketones homogeneously or heterogeneously mixed in it.

Preferably, the Ketone Oral Transfer Device saliva permeable carrier comprises a saliva permeable pouch.

The pouch, solid strip, or lozenge may have pores or fissures through which the edible ketones in the pouch can seep dissolved in saliva.

The pouch may comprise a fabric or a textile. It may comprise a fibrous material. The pouch may be made from a material, thin, like a membrane. A membrane permeable to water and oral saliva.

Pores in the pouch may formed by piercing the pouch with fine needles. The pores may be formed by a weave or tangling of threads or fibers which the pouch material comprises.

Preferably, the saliva permeable carrier may be circular, oval, or rectangular. The carrier may be substantially strip shaped or lozenge shaped. It may have a longest dimension of 0.5 cm, 1.0 cm, 1.5 cm, or 2 cm. Preferably, the length of the longest dimension is at least 0.5 cm. It may be at most 2.5 cm. Preferably the thickness of the carrier is at least 0.05 cm. Preferably the thickness is at most 0.5 cm or 1 cm, so, the pouch may rest on a person's tongue or between cheek and gum.

Preferably, the pouch is dissolvable in saliva to gradually dissolve away as the edible ketones in the pouch are dissolving. So when both the pouch and ketones have dissolved there is nothing the person must spit and throw away.

Preferably, the pouch or solid strip may comprise a material which is solid at room temperature up to at least twenty-five degrees C. The material may have a melting point a few degrees below the normal oral temperature. Preferably, the material melts at a temperature between twenty-seven degrees C. and thirty-seven degrees C.

As the pouch or solid strip melts in the mouth due to dissolving of the pouch material in saliva or melting of the pouch material in the warm mouth, pores may form and/or may get larger. Thus initially the ketones seep out of the pouch slowly or not all. As the pores increase in number and size, more saliva and ketones are let through.

Preferably, the pouch or solid strip comprises a material which is tasteless. So the major taste a person experiences is that of the ketones dissolved in the saliva or a flavoring included in the pouch. Preferably, the flavoring is mixed in the pouch.

The edible ketones may have a powdered form in the pouch or mixed/embedded into the solid strip.

The edible ketones may be in a gel, gelatin, or gelatinized form. The gel may dissolve in saliva. Preferably the gel is contained in the pouch and in the gel form cannot leak through pores or fissures in the pouch until dissolved by saliva. The gel may dissolve in saliva. Then the ketones may seep out of the pouch with the saliva.

An advantage of the ketone oral transfer device is its portability and adjustability in dosing. Any size dose can be initially placed in a ketone oral transfer device. Further, by increasing or decreasing the time a ketone oral transfer device is retained in a mouth, the amount of a dose present in a ketone oral transfer device can be used for a duration of time from minimal time only due to contact with the device through oral transfer or maximum from complete consumption of the dose present in the device.

The invention will now be described, by way of example only, with reference to the accompanying drawings in which:

### BRIEF DESCRIPTION OF THE DRAWINGS

FIG. 1 is a perspective view of the liquid soluble ketone oral transfer device with ketone inside according to the invention;

FIG. 2 is a perspective view of the liquid soluble ketone oral transfer device without ketone inside according to the invention; and

FIG. 3 is a perspective view of the liquid soluble membrane in the form of a flat solid strip containing ketones according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

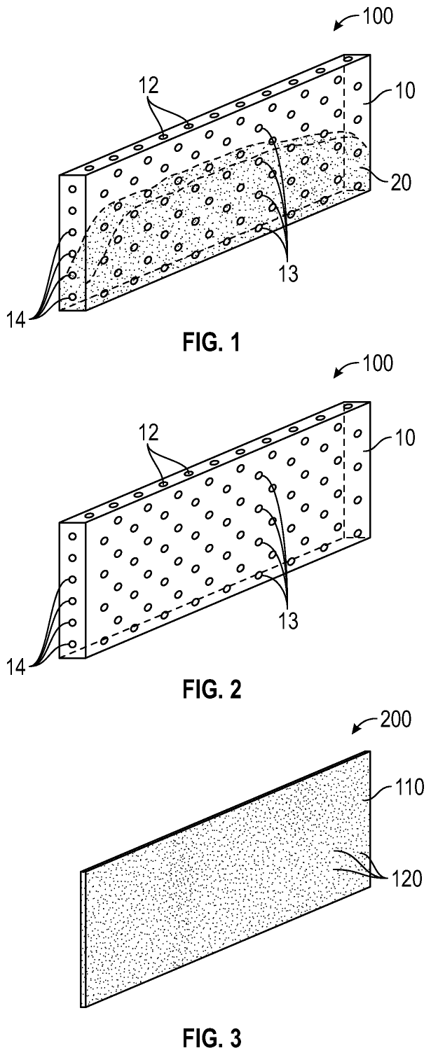

Referring to the Figures, there is shown in FIG. 1 an Element 100, which is a liquid permeable ketone oral transfer device. The ketone oral transfer device (100) comprises a saliva permeable carrier, Element 10, in the form of a pouch which contains an edible ketone substance, Element 20, which is soluble in liquid saliva,

A person can hold the carrier pouch (10) on their tongue or between their cheek and gum. The carrier pouch (10) has pores to allow liquid saliva to seep into and out of the carrier pouch (10). As shown in FIG. 1, Elements 12, 13, and 14 are pores in the carrier pouch (10) which render the carrier pouch (10) permeable to liquid saliva. The carrier pouch (10) has front and back faces, and there are pores, for example pores 13, for liquid saliva to seep through both the front and back faces. The front and back faces are connected by side walls, and there are pores, for example pores (12) and pores (14), through the side walls.

In one embodiment, the edible ketones (20) in the carrier pouch (110) are a powder. The powder is a pure edible ketone or a mixture with another powder which dilutes the concentration of ketones in the pouch (10) for more gradual dissolving and release of ketones to the user. The grains of the powder are larger than the pores to prevent edible ketone release except by dissolving in saliva.

In another embodiment the edible ketones (20) in the carrier strip (110) are in a gel, gelatin, or are gelatinized. Factors such as viscosity and/or surface tension of the gel prevent it from seeping through the pores (12, 13, and 14) until the gel/gelatin is dissolved in saliva.

Referring to the Figures, there is shown in FIG. 2 an Element 10 which is an empty liquid permeable ketone carrier pouch, and, Elements 12, 13, and 14 which are pores in the carrier pouch (10) and which provide the carrier pouch (10) with liquid saliva permeability.

The carrier pouch (10) is made of a material which is a cloth or paper-like material or a polymeric film. In one embodiment, the material is dissolvable in liquid saliva so the carrier pouch (10) is dissolved and consumed by the user as the edible ketones are dissolved by saliva. In another embodiment the carrier pouch (10) is not dissolvable and impervious to liquid saliva. So the carrier pouch may be refilled and re-used.

The walls of the carrier pouch (10) are membranes through which saliva is permeable. The pores (12, 13, and 14) in the carrier pouch (10) shown in FIG. 1 and FIG. 2 are perforations through the carrier pouch (10). Alternatively, the pores could be spaces between threads in a weave of a fabric, cloth, or a paper-like material.

The carrier pouch (10) may have a re-sealable opening to receive the edible ketones (20). The edible ketone (20) may be replenished within the carrier pouch (10) by unsealing the opening of the carrier pouch (10), putting more edible ketone (20) into the carrier pouch (10) and resealing the opening.

Referring to the Figures, there is shown in FIG. 3 an Element 200 which is another embodiment of the ketone oral transfer device. As shown in FIG. 3, the device (200) comprises an element (110) which is a solid strip comprising edible ketones (120) which are soluble in saliva. Element (120), said edible ketones, is a liquid soluble ketone substance embedded in the liquid permeable membrane (110). The solid strip (110) comprises a flexible membrane which a person may hold on their tongue or between their cheek and gum.

The edible ketones (120) are embedded as a heterogeneous mixture in a substrate of the solid strip (110). For example, the edible ketones (120) are particulates of a powder embedded in the solid strip (110). Alternatively the edible ketones are homogeneously mixed or dissolved in solid strip (110).

In an embodiment, the solid strip (110) is itself soluble in saliva to release the edible ketones (120). The released ketones (120) then in turn dissolve in the saliva. Alternatively, the solid strip (110) may be in-dissolvable and/or impervious to the saliva. The solid strip (110) may be porous to facilitate access of the liquid saliva to more surface area of the edible ketones (120) which are either heterogeneously or homogeneously mixed in the solid strip (110).

The device (100) of the present invention comprises a liquid permeable ketone oral transfer device (10) containing liquid soluble ketones (20) that can be placed between the cheek and gums for the purpose of ingesting or absorbing the ketones contained therein for usual consumption, or to enable an increase in energy.

An alternative embodiment of the device of the present invention comprises a liquid soluble membrane solid strip containing liquid soluble ketones (200) that can be placed between the cheek and gums, or on the tongue for the purpose of ingesting or absorbing the ketones contained therein for usual consumption, or to enable an increase in energy.

Using the preferred embodiment, or an alternate embodiment, there is a method of ingesting ketones for dietary consumption using a liquid permeable ketone oral transfer device containing liquid soluble ketones (100), wherein said ketone oral transfer device comprises a carrier pouch (10) which is placed between a cheek and a gum line, and left in that position for a length of time to enable an amount of an oral liquid soluble ketone substance (20) to be dissolved into an amount of saliva excreted by a mouth during the time said ketone oral transfer device is in position between the cheek and gum. There is also an alternate method of ingesting ketones to increase a person's energy level using a liquid permeable ketone oral transfer device containing liquid soluble ketones (100), wherein said ketone oral transfer device is placed between a cheek and a gum line, and left in that position for a length of time to enable an amount of a liquid soluble ketone substance (20) to be dissolved into an amount of saliva excreted by a mouth during the time said ketone oral transfer device is in position between the cheek and gum.

Using the preferred embodiment, or an alternate embodiment, there is a method of ingesting ketones for dietary consumption using a liquid soluble membrane strip containing liquid soluble ketones 200, wherein said solid strip (110) is placed between a cheek and a gum line, and left in that position for a length of time to enable an amount of a liquid soluble ketone substance (20) to be dissolved into an amount of saliva excreted by a mouth during the time said solid strip is in position between the cheek and gum. There is also an alternate method of ingesting ketones to increase a person's energy level using a liquid soluble membrane solid strip (110) containing liquid soluble ketones 200, wherein said solid strip (110) is placed between a cheek and a gum line, and left in that position for a length of time to enable said solid strip to be dissolved into an amount of saliva excreted by a mouth during the time said solid strip is in position between the cheek and gum.

Using the preferred embodiment, or an alternate embodiment, there is a method of ingesting ketones for dietary consumption using a liquid soluble membrane solid strip (110) containing liquid soluble ketones 200, wherein said solid strip (110) is placed a tongue, and left in that position for a length of time to enable dissolving of said solid strip into a mouth wherein said solid strip is dissolved by an amount of saliva excreted by a mouth during the time said solid strip is in position on said tongue. There is also an alternate method of ingesting ketones to increase a person's energy level using a liquid soluble membrane solid strip containing liquid soluble ketones 200, wherein said solid strip is placed on a tongue, and left in that position for a length of time to enable said solid strip to be dissolved into the mouth for ingestion or absorption enabled by an amount of saliva excreted by a mouth during the time said solid strip is in position on said tongue.

In a preferred embodiment, there is a ketone oral transfer device comprising an orally retainable carrier in a form of a pouch containing an edible ketone arranged to seep out of the pouch in an oral saliva.

In a preferred embodiment, there is a ketone oral transfer device comprising an orally retainable carrier in a form of a solid strip comprising an edible ketone arranged to seep out of the solid strip in an oral saliva.

In a preferred embodiment, there is a ketone oral transfer device wherein the carrier has pores or fissures through which the edible ketones mixed with said oral saliva can seep out.

In a preferred embodiment, there is a ketone oral transfer device wherein the pouch comprises a membrane permeable to said oral saliva and the edible ketone in the oral saliva.

In a preferred embodiment, there is a ketone oral transfer device wherein the edible ketone is heterogeneously mixed as particles in the solid strip.

In a preferred embodiment, there is a ketone oral transfer device wherein the edible ketone is homogeneously dissolved in the solid strip.

In a preferred embodiment, there is a ketone oral transfer device wherein the carrier has a longest dimension which is at least 0.5 cm and at most 2.5 cm.

In a preferred embodiment, there is a ketone oral transfer device wherein the carrier has a thickness which is at least 0.05 cm and at most 1 cm.

In a preferred embodiment, there is a ketone oral transfer device wherein the pouch is solid up to at least twenty-five degrees C. and melts at or below a normal oral temperature.

In a preferred embodiment, there is a ketone oral transfer device wherein the edible ketone is solid up to at least twenty-five degrees C. and melts at or below a normal oral temperature.

In a preferred embodiment, there is a ketone oral transfer device wherein said orally retainable carrier is tasteless.

In an alternate embodiment, there is a ketone oral transfer device wherein the edible ketone is stored in a mixture comprising a flavoring which dissolves in said oral saliva.

In an alternate embodiment, there is a ketone oral transfer device wherein the edible ketone is a gel or gelatin.

In a preferred embodiment, there is a ketone oral transfer device wherein said edible ketone is a powder.

An advantage of the ketone oral transfer device is its portability and adjustability in dosing. Any size dose can be initially placed in a ketone oral transfer device. Further, by increasing or decreasing the time a ketone oral transfer device is retained in a mouth, the amount of a dose present in a ketone oral transfer device can be used for a duration of time from minimal time only due to contact with the device through oral transfer or maximum from complete consumption of the dose present in the device.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A ketone oral transfer device comprising an orally retainable carrier in a form of a pouch containing an edible ketone arranged to seep out of the pouch in an oral saliva.

2. A ketone oral transfer device comprising an orally retainable carrier in a form of a solid strip comprising an edible ketone arranged to seep out of the solid strip in an oral saliva.

3. The ketone oral transfer device according to claim 1 wherein the carrier has pores or fissures through which the edible ketones mixed with said oral saliva can seep out.

4. The ketone oral transfer device according to claim 2 wherein the carrier has pores or fissures through which the edible ketones mixed with said oral saliva can seep out.

5. The ketone oral transfer device according to claim 1 wherein the pouch comprises a membrane permeable to said oral saliva and the edible ketone in the oral saliva.

6. The ketone oral transfer device according to claim 2 wherein the edible ketone is heterogeneously mixed as particles in the solid strip.

7. The ketone oral transfer device according to claim 2 wherein the edible ketone is homogeneously dissolved in the solid strip.

8. The ketone oral transfer device according to claim 1 wherein the carrier has a longest dimension which is at least 0.5 cm and at most 2.5 cm.

9. The ketone oral transfer device according to claim 2 wherein the carrier has a longest dimension which is at least 0.5 cm and at most 2.5 cm.

10. The ketone oral transfer device according to claim 1 wherein the carrier has a thickness which is at least 0.05 cm and at most 1 cm.

11. The ketone oral transfer device according to claim 2 wherein the carrier has a thickness which is at least 0.05 cm and at most 1 cm.

12. The ketone oral transfer device according to claim 1 wherein the pouch is solid up to at least twenty-five degrees C. and melts at or below a normal oral temperature.

13. The ketone oral transfer device according to claim 2 wherein the solid strip is solid up to at least twenty-five degrees C. and melts at or below a normal oral temperature.

14. The ketone oral transfer device according to claim 1 wherein the edible ketone is solid up to at least twenty-five degrees C. and melts at or below a normal oral temperature.

15. The ketone oral transfer device according to claim 1 wherein said orally retainable carrier is tasteless.

16. The ketone oral transfer device according to claim 2 wherein said orally retainable carrier is tasteless.

17. The ketone oral transfer device according to claim 1 wherein the edible ketone is stored in a mixture comprising a flavoring which dissolves in said oral saliva.

18. The ketone oral transfer device according to claim 1 wherein the edible ketone is a gel or gelatin.

19. The ketone oral transfer device according to claim 1 wherein said edible ketone is a powder.
